(19) United States
(12) Patent Application Publication
(10) Pub. No.: US 2021/0235738 A1
(43) Pub. Date: Aug. 5, 2021

(54) PALATINOSE SYRUP HAVING INHIBITED CRYSTALIZATION AND HAVING ABILITY TO SUPPRESS BLOOD SUGAR LEVEL INCREASE

(71) Applicant: NEO CREMAR CO.,LTD, Seoul (KR)

(72) Inventors: Jae-Hwan KIM, Seoul (KR); Na-Ri KIM, Seoul (KR)

(21) Appl. No.: 17/049,303

(22) PCT Filed: Nov. 23, 2018
(86) PCT No.: PCT/KR2018/014562

§ 371 Date: Oct. 20, 2020

(30) Foreign Application Priority Data
Apr. 20, 2018 (KR) 10-2018-0046376

(51) Int. Cl. A23L 33/125; A61K 31/7016; A61K 31/7004

(52) U.S. Cl. CPC A23L 33/125; A61K 31/7004; A61K 31/7016

(57) ABSTRACT

The present invention relates to palatinose syrup comprising palatinose, glucose, fructose and trehalulose, the palatinose syrup comprising 19˜22 wt % of palatinose with respect to the total amount of solids. Further, the present invention relates to a medicine or food product comprising the palatinose syrup.

### TECHNICAL FIELD

The present invention relates to palatinose syrup.

### BACKGROUND ART

Palatinose is used as a sugar substitute because it has an effect of inhibiting the elevation of blood sugar level, compared to sugar.

For example, Korean Patent No. 10-1450786 discloses a method for producing an agar jelly using palatinose, and Korean Patent No. 10-1087000 discloses a method for improving the flavor of a fermented soybean milk using palatinose.

However, palatinose has been typically used in the form of a solid, a powder, or the like, because it is difficult to use palatinose in a liquid form such as syrup due to a low solubility thereof.

When palatinose is applied to foods and medicines, there are some cases where a liquid form is preferred to a solid form, and, thus, there has been attempts to formulate palatinose into syrup. However, an existing palatinose syrup has a problem of precipitation of crystals when stored at room temperature or under a refrigerated condition (for example, −4° C.) for a certain period of time, for example, for 4 months or more.

Accordingly, the present inventors provide a method for producing palatinose syrup which is excellent in inhibiting the precipitation of crystals and the elevation of blood sugar level.

### DISCLOSURE

### Technical Problem

It is an object of the present invention to provide palatinose syrup which has a high solubility, inhibits precipitation of a crystal, and has an inhibition activity of the elevation of blood sugar level.

### Technical Solution

To achieve the above object, the present invention provides palatinose syrup comprising palatinose, glucose, fructose and trehalulose, wherein the palatinose is present in an amount of 19 wt % to 22 wt %, based on the total solid contents.

### Advantageous Effects

Palatinose syrup of the present invention has a high solubility, inhibits precipitation of a crystal, and has an inhibition activity of the elevation of blood sugar level.

### DESCRIPTION OF DRAWINGS

FIG. 1 is a photograph showing the solubility of xylitol, white sugar, glucose hydrocrystalline, crystalline fructose and palatinose (xylitol, white sugar, glucose hydrocrystalline, crystalline fructose, and palatinose in order from left to right in the drawing).

FIG. 2 shows the blood glucose concentration per hour measured by oral glucose tolerance test when palatinose is administered in variety of an amount.

FIG. 3 shows the result of calculating the incremental area under curve (AUC) between 0 minute and 180 minutes after administration of the sample through oral glucose tolerance test when palatinose is administered in variety of an amount.

FIG. 4 shows the result of measuring blood glucose concentration per hour through oral glucose tolerance test when palatinose syrups having different composition ratios are administered.

FIG. 5 shows the result of calculating the incremental area under curve (AUC) between 0 minute and 180 minutes after the administration of the sample through the oral glucose tolerance test when palatinose syrups having different composition ratios are administered.

FIG. 6 shows the results of observing whether or not a crystal of the palatinose syrup is formed at room temperature.

FIG. 7 shows the results of observing whether or not a crystal of the palatinose syrup is formed at low temperature.

### BEST MODE

The present invention relates to palatinose syrup comprising palatinose, glucose, fructose and trehalulose, wherein the palatinose is present in an amount of 19 w% to 22 wt %, based on the total solid contents.

Further, the present invention is directeds to a food composition for inhibiting the elevation of blood sugar level, which comprises the palatinose syrup of the present invention.

Hereinafter, the present invention will be described in detail.

### Palatinose Syrup

The palatinose syrup of the present invention comprises palatinose, glucose, fructose, and trehalulose, wherein the palatinose is contained in an amount of 19 wt % to 22 wt % based on the total solid contents. The palatinose syrup of the present invention may have a sugar content from 69° to 78° BRIX. Further, the palatinose syrup of the present invention does not contain crystals, precipitates and floaters at room temperature. Furthermore, the palatinose syrup of the present invention does not produce crystals, precipitates and floaters even when stored at room temperature or at a low temperature of −4° C. for 6 months. In addition, the palatinose syrup of the present invention has an inhibition activity of the elevation of blood sugar level.

In one embodiment of the present invention, the palatinose syrup of the present invention comprises 80 to 250 parts by weight of glucose, 80 to 250 parts by weight of fructose, and 0.3 to 12 parts by weight of trehalulose, based on 100 parts by weight of palatinose. For example, the palatinose syrup of the present invention may contain 20 wt % to 22 wt % of palatinose, 20 wt % to 45 wt % of glucose, 20 wt % to 45 wt % of fructose, and 0.1 wt % to 2.0 wt % of trehalulose, based on the total solid contents.

In one embodiment of the present invention, the palatinose syrup of the present invention can be prepared according to the following method: 300 g to 500 g of sugar are added to 500 ml to 700 ml of water and dissolved with stirring at 70° C. to 80° C. to obtain an aqueous sugar solution. The pH of the dissolved sugar solution is adjusted to pH 5-pH 7. 0.01 wt % to 0.5 wt % of alpha-glucosyl transferase is added based on the solid contents of the aqueous sugar solution, and an enzyme reaction is carried out at 30° C. to 40° C. for 15 to 30 hours. After the enzyme reaction is completed, the enzyme is inactivated at 80° C. to 90° C. for 30 minutes. 0.01 wt % to 0.1 wt % of invertase is added and mixed, based on the solid contents of the inactivated reaction solution, and then, the mixture is reacted at 50° C. to 60° C. for 2 to 24 hours. After the enzyme reaction is completed, the enzyme is inactivated at 80° C. to 90° C. for 30 minutes. An activated carbon is added to the mixture in an amount of 0.1 wt % to 0.5 wt %, based on the solid contents, and then reacted at 60° C. to 70° C. for 2 hours. Thereafter, the mixture is filtered with a 0.5 pm filter followed by being passed through a 0.22 μm filter finally, and then, concentrated to 68°-78° Brix to prepare palatinose syrup. The palatinose syrup prepared in this way contains palatinose, glucose, fructose and trehalulose.

In one embodiment of the present invention, the palatinose syrup of the present invention may further comprise sugar. Preferably, the palatinose syrup of the present invention may comprise palatinose, sugar, glucose, fructose and trehalulose. In this case, the palatinose syrup of the present invention may contain 300-420 parts by weight of sugar, 5-21 parts by weight of fructose, and 5-21 parts by weight of glucose, based on 100 parts by weight of palatinose. For example, the palatinose syrup of the present invention may contain 20 wt % to 22 wt % of palatinose, 70 wt % to 78.5 wt % of sugar, 1.0 wt % to 3.5 wt % of fructose, 1.0 wt % to 3.5 wt % of glucose, 0.1 wt % to 2.0 wt % of trehalulose, based on the total solid contents.

In one embodiment of the present invention, the palatinose syrup of the present invention can be prepared according to the following method: 300 g to 500 g of sugar is added to 500 ml to 700 ml of water and dissolved with stirring at 70° C. to 80° C. to obtain an aqueous sugar solution. The pH of the dissolved sugar solution is adjusted to pH 5 to pH 7. 0.01 wt % to 0.5 wt % of alpha-glucosyl transferase is added, based on the solid contents of the aqueous sugar solution, and an enzyme reaction is carried out at 30° C. to 40° C. for 15 to 30 hours. After the enzyme reaction is completed, the enzyme is inactivated at 80° C. to 90° C. for 30 minutes. Then, an activated carbon is added in an amount of 0.1 wt % to 0.5 wt %, based on the solid contents, and then, reacted at 60° C. to 70° C. for 2 hours. Thereafter, the reaction mixture is filtered with a 0.5 μm filter followed by being passed through a 0.22 μm filter finally, and then concentrated to 68°-78° Brix to prepare palatinose syrup. The palatinose syrup prepared in this way contains palatinose, sugar, glucose, fructose and trehalulose.

In one embodiment of the invention, the palatinose syrup of the present invention may further comprise a fructooligosaccharide. Preferably, the palatinose syrup of the present invention may comprise palatinose, fructooligosaccharide, sugar, glucose, fructose and trehalulose. In this case, the palatinose syrup of the present invention may contain 65-170 parts by weight of fructooligosaccharide, 65-170 parts by weight of sugar, 65-140 parts by weight of glucose, 20-70 parts by weight of fructose, and 0.3-12 parts by weight of trehalulose, based on 100 parts by weight of palatinose. For example, the palatinose syrup of the present invention may contain 20 wt % to 22 wt % of palatinose, 15 wt % to 30 wt % of fructooligosaccharide, 15 wt % to 30 wt % of sugar, 15 wt % to 25 wt % of glucose, 5 wt % to 15 wt % of fructose, 0.1 wt % to 2.0 wt % of trehalulose, based on the total solid contents.

In one embodiment of the present invention, the palatinose syrup of the present invention can be prepared according to the following method: 300 g to 500 g of sugar is added to 500 ml to 700 ml of water and dissolved with stirring at 70° C. to 80° C. to obtain an aqueous sugar solution. The pH of the dissolved sugar solution is adjusted to pH 5 to pH 7. 0.01 wt % to 0.5 wt % of alpha-glucosyl transferase is added, based on the solid contents of the aqueous sugar solution, and an enzyme reaction is carried out at 30° C. to 40° C. for 15 to 30 hours. After the enzyme reaction is completed, the enzyme is inactivated at 80° C. to 90° C. for 30 minutes. 0.1 wt % to 1.5 wt % of fructosyltransferase is added and mixed, based on the solid contents of the inactivated reaction solution, and the mixture is reacted at 50° C. to 60° C. for 24 to 72 hours. After the enzyme reaction is completed, the enzyme is inactivated at 80° C. to 90° C. for 30 minutes. Thereafter, an activated carbon is added in an amount of 0.1 wt % to 0.5 wt %, based on the solid contents, and then, reacted at 60° C. to 70° C. for 2 hours. Then, the reaction mixture is filtered with a 0.5 μm filter followed by being passed through a 0.22 μm filter finally, and then, concentrated to 68°-78° Brix to prepare palatinose syrup. The palatinose syrup prepared in this way contains palatinose, fructooligosaccharide, sugar, glucose, fructose and trehalulose.

### Food Composition

The present invention relates to a food composition for inhibiting the elevation of blood glucose level, comprising the palatinose syrup of the present invention. The food of the present invention may be a health supplement, a health functional food, a functional food, and the like, but is not limited thereto, and may comprise a composition of the present invention mixed with a natural food, a processed food, a general food material, and the like.

A food composition of the present invention may be used as the palatinose syrup of the present invention as it is, or in combination with other food or food composition, and may be suitably used according to a conventional method. The amount of the active ingredients to be mixed can be suitably determined depending on its intended use (prevention, improvement or therapeutic treatment). In general, the palatinose syrup of the present invention may be added in an amount of 0.1 wt % to 70 wt %, preferably 2 wt % to 50 wt %, based on 100 wt % of the raw material of food or beverage when the food or the beverage is produced. The effective dose of the palatinose syrup of the present invention can be used in accordance with the effective dose of the pharmaceutical composition. However, in the case of ingesting for the purpose of health and hygiene or taking a long-term intake for the purpose of controlling health, it may be lower than the above range. In addition, since the active ingredient of palatinose syrup of the present invention has no problem in terms of safety, it can be used in an amount exceeding the above range.

There is no particular limitation on the kind of the food. The food composition may be used in the form of an oral administration preparation such as tablets, hard or soft capsules, liquids, suspensions, and the like. In addition, these preparations can be prepared using conventionally acceptable carriers, for example, excipients, binders, disintegrating agents, glidants, solubilizers, suspending agents, preservatives or extenders, in the case of oral administration preparations.

Examples of the foods to which the palatinose syrup of the present invention can be added include meat products, sausages, breads, chocolates, candies, snack products, confectionery, pizza, ramen, other noodles, gums, dairy products including ice cream, various soup, beverage, tea, a drink, an alcoholic beverage and a vitamin complex, and other nutrients, but the present invention is not limited to these kinds of foods.

For example, the palatinose syrup of the present invention can be used for the production of frozen dough for baking. In the case of producing frozen dough for baking using the palatinose syrup of the present invention and producing the bread using the frozen dough, the destruction of the yeast due to the ice crystal growth is minimized, after baking, the volume reduction of the bread is suppressed, and the moisture content of the bread is increased. Therefore, it is possible to provide bread having a soft and moist mouthfeel.

### Pharmaceutical Composition

The present invention relates to a pharmaceutical composition for suppressing the elevation of blood sugar level, which comprises the palatinose syrup of the present invention. The pharmaceutical composition of the present invention can be administered to a patient diagnosed as diabetes, an ordinary person who has to suppress blood glucose, or a person or a mammal who is at risk of diabetes or who is to be prevented from diabetes. The pharmaceutical composition may be a pharmaceutical composition for preventing or treating diabetes.

The pharmaceutical composition of the present invention may contain 0.01 wt % to 80 wt %, preferably 0.02 wt % to 65 wt %, of the palatinose syrup. However, this amount can be increased or decreased depending on the needs of a person who takes medicine and can be appropriately increased or decreased depending on the conditions such as dietary life, nutritive condition, degree of disease progression, and degree of obesity.

The pharmaceutical composition of the present invention can be administered orally or parenterally and can be used in the form of a general pharmaceutical preparation. Preferred pharmaceutical preparations are preparations for oral administration, such as tablets, hard or soft capsules, liquids, suspensions, etc. These pharmaceutical preparations can be prepared using pharmaceutically acceptable carriers, for example, excipients, binders, disintegrating agents, glidants, solubilizers, suspending agents, preservatives or extenders, in the case of preparations for an oral administration.

The dosage of the pharmaceutical composition comprising the palatinose syrup of the present invention can be determined by a specialist according to various factors such as the condition of a patient, age, gender, and complication. In general, however, it may be administered in a dose of 0.1 mg to 10 g, preferably 10 mg to 5 g, per 1 kg of the adult body weight. The dosage of the pharmaceutical composition may be a daily dose, a half dose, ⅓ dose or ¼ dose, and it may be administered 1 times to 6 times a day. However, in the case of ingesting for the purpose of health and hygiene or taking a long-term intake for the purpose of controlling health, it may be lower than the above range. In addition, since the active ingredient of the palatinose syrup of the present invention has no problem in terms of safety, it can be used in an amount exceeding the above range.

Advantages and features of the present invention, and methods for accomplishing the same will become apparent with reference to the examples described below in detail. It should be understood, however, that the present invention is not limited to the examples disclosed herein, but may be implemented in many different types, and should not be construed as limited to the examples set forth herein. These examples are provided to fully disclose the scope of the invention to persons skilled in the relevant art. Further, the invention is only defined by the scope of the claims described below.

### Materials and Methods

Commercially available products such as xylitol, white sugar, glucose hydrocrystalline, crystalline fructose, palatinose, alpha-glucosyl transferase, invertase, and fructosyltransferase were purchased and used as the materials of the present invention.

### EXAMPLE 1

500 g of sugar was added to 700 ml of water and dissolved with stirring at 75° C. to obtain an aqueous sugar solution. The pH of the dissolved sugar solution was adjusted to pH 6. Then, 0.3 wt % of α-glucosyl transferase was added based on the solid contents of the aqueous sugar solution, and an enzyme reaction was carried out at 30° C. for 20 hours. After completion of the enzyme reaction, the enzyme was inactivated at 90° C. for 30 minutes, and 0.5 wt % of activated carbon was added based on the solid contents, and then reacted at 65° C. for 2 hours. Thereafter, the reaction product was filtered using a 0.5 μm filter followed by being passed through a 0.22 μm filter finally, and then concentrated to 70° Brix to prepare palatinose syrup of Example 1. The obtained palatinose syrup had 20 wt % of palatinose, 76 wt % of sugar, 1.3 wt % of fructose, 1.6 wt % of glucose and 1.1 wt % of trehalulose, based on the total solid contents.

### EXAMPLE 2

500 g of sugar was added to 700 ml of water and dissolved with stirring at 75° C. to obtain an aqueous sugar solution. The pH of the dissolved sugar solution was adjusted to pH 6. Then, 0.3 wt % of α-glucosyl transferase was added based on the solid contents of the aqueous sugar solution, and an enzyme reaction was carried out at 30° C. for 20 hours. After the completion of the enzyme reaction, the enzyme was inactivated at 90° C. for 30 minutes, and 0.1 wt % of the invertase was added and mixed, based on the solid contents of the inactivated reaction solution. Then, the mixture was reacted at 55° C. for 20 hours, and then, inactivated at 90° C. for 30 minutes. 0.5 wt % of activated carbon was added based on the solid contents, and reacted at 65° C. for 2 hours. Thereafter, the reaction product was filtered using a 0.5 μm filter followed by being passed through a 0.22 μm filter finally, and then concentrated to 70° Brix to prepare palatinose syrup of Example 2. The palatinose syrup (palatinose IS) prepared in this way had 20 wt % of palatinose, 37.1 wt % of fructose, 41.4 wt % of glucose and 1.5 wt % of trehalulose, based on the total solid contents.

### EXAMPLE 3

500 g of sugar was added to 700 ml of water and dissolved with stirring at 75° C. to obtain an aqueous sugar solution. The pH of the dissolved sugar solution was adjusted to pH 6. Then, 0.3 wt % of α-glucosyl transferase was added based on the solid contents of the aqueous sugar solution, and the enzyme reaction was carried out at 30° C. for 20 hours. After the completion of the enzyme reaction, the enzyme was inactivated at 90° C. for 30 minutes, and 0.7 wt % of the fructosyltransferase was added and mixed, based on the solid contents of the inactivated reaction solution. Then, the mixture was reacted at 55° C. for 58 hours, and then inactivated at 90° C. for 30 minutes. 0.5 wt % of activated carbon was added based on the solid contents, and reacted at 65° C. for 2 hours. Thereafter, the reaction product was filtered using a 0.5 μm filter followed by being passed through a 0.22 μm filter finally, and then concentrated to 70° Brix to prepare palatinose syrup of Example 3. The palatinose syrup (palatinose FOS) prepared in this way had 20 wt % of palatinose, 22 wt % of fructooligosaccharide, 24 wt % of sugar, 10 wt % of fructose, 22.7 wt % of glucose and 1.3 wt % of trehalulose, based on the total solid contents.

### Comparative Example 1

840 g of sugar was added to 350 ml of water and dissolved with stirring at 50° C. to obtain an aqueous sugar solution. Then, 10 g of alpha-glucosyl transferase was added to the sugar solution and adjusted to pH 6 to 7 by adding 0.1% of sodium hydroxide aqueous solution. After 5 hours from the initiation of the reaction, 6 g of palatinose crystals were added to initiate the crystallization reaction, wherein the palatinose crystals had seed crystals with an average particle diameter of 100 μm. After 28 hours from the initiation of the reaction, the stirring was stopped to complete the reaction. At this time, the state of reaction solution was in a slurry state and the sucrose contents was 0.1 wt %. This slurry state of the reaction solution was dehydrated by putting it in a centrifugal dehydrator (the concentration of solid contents 93.5 wt %, purity 98.6%). 20 wt % of the dehydrated palatinose was dissolved in 80 wt % of water, and then concentrated to 70° Brix.

### Experimental Example 1

The solubility of sweeteners was evaluated. Specifically, 90 wt % of distilled water was added to each of 10 wt % of sweeteners such as xylitol, white sugar, glucose hydrocrystalline, crystalline fructose and palatinose, and the mixture was dissolved in a mixer at 40 rpm for 1 minute.

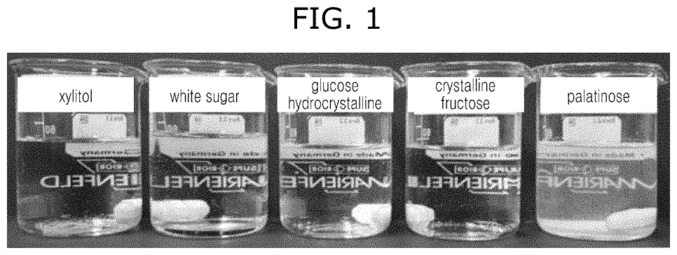

As a result, it has been confirmed that xylitol, white sugar, glucose hydrocrystalline and crystalline fructose had excellent solubility in view of the fact that no precipitates or floaters were observed. However, it was observed that palatinose was little dissolved and floated (see FIG. 1, respectively xylitol, white sugar, glucose hydrocrystalline, crystalline fructose and palatinose from left to right).

### Experimental Example 2

The effect of inhibiting the elevation of blood sugar level was evaluated depending on the palatinose contents. For the evaluation, 1) syrup containing 20 wt % of palatinose, 2) syrup containing 25 wt % of palatinose, 3) syrup containing 30 wt % of palatinose, 4) syrup containing 100 wt % of palatinose , 5) sugar syrup containing 100 wt % of sugar, and 6) glucose syrup containing 100 wt % of glucose, were prepared on the basis of the total solid contents, respectively. At this time, the syrups of 1) to 3) contained the solid contents of fructose, glucose, sugar and trehalulose, except for palatinose, and each of the syrups contained the same amounts of fructose, glucose, sugar and trehalulose. Further, the syrups of 1) to 6) used water as a liquid component, except for the solid contents.

Oral glucose tolerance test (OGTT) was performed on the syrups of 1) to 6). Specifically, 6-week-old female BALB/c mice were starved for 12 hours, and a basal blood glucose was measured. Thereafter, each of the samples was ingested in a concentration of 2 mg/g, and the blood glucose concentration of the blood collected at 0 minute (before the samples are administered), and 30 minutes, 60 minutes, 90 minutes, 120 minutes and 180 minutes after the administration of the samples was measured with a glucometer.

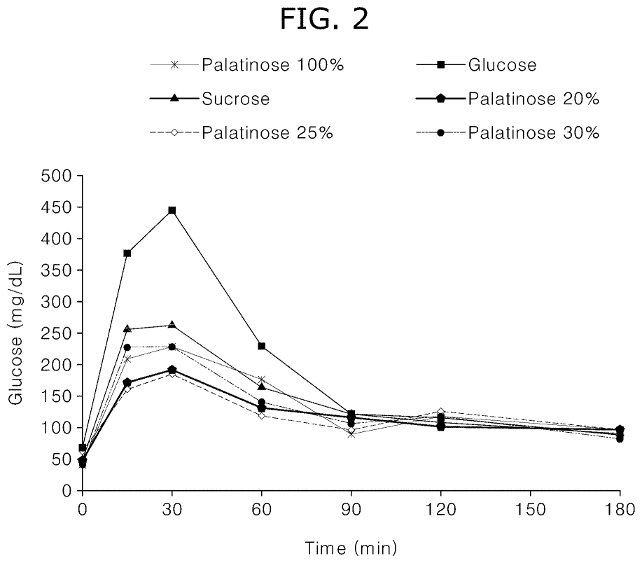

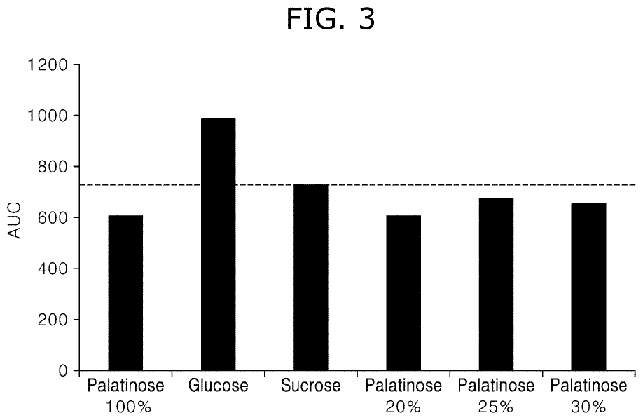

The blood glucose concentration at each time was indicated by a line in the graph and the incremental area under curve (AUC) between 0 and 180 minutes was calculated. As a result, it was confirmed that the syrup 1) containing 20 wt % of palatinose had an effect of inhibiting the elevation of blood sugar level similar to that of the syrup 4) containing 100 wt % of palatinose. However, the syrup 2) containing 25 wt % of palatinose and the syrup 3) containing 30 wt % of palatinose had a lower effect of inhibiting the elevation of blood sugar level than the syrup 1) containing 20 wt % of palatinose (see FIGS. 2 and 3). This result is not consistent with the expectation that the concentration of palatinose will rely on an effect of inhibiting the elevation of blood sugar level.

### Experimental Example 3

It has been confirmed from Experimental Example 2 that the syrup containing 20 wt % of palatinose had an effect of inhibiting the elevation of blood sugar level similar to that of the syrup containing 100 wt % of palatinose. Therefore, syrups keeping the contents of palatinose to 20 wt %, and having different sugar composition ratios were prepared, respectively, to evaluate the inhibitory effect of blood sugar level for these syrups.

As samples, palatinose syrups of Examples 1 to 3, distilled water (DW) and sucrose were used to perform Oral glucose tolerance test (OGTT). Six-week-old female BALB/c mice were dosed with each of the samples and their changes in blood sugar levels were measured. First, mice were starved for 12 hours, and then a basal blood glucose was measured. Thereafter, each of the samples was ingested in a concentration of 2 mg/g, and the blood glucose concentration of the blood collected at 0 minute (before the samples are administered), 30 minutes, 60 minutes, 90 minutes, and 120 minutes after the administration of the samples was measured with a glucometer.

The blood glucose concentration at each time after the administration of the sample was indicated by a line in the graph and the incremental area under curve (AUC) between 0 and 120 minutes was calculated.

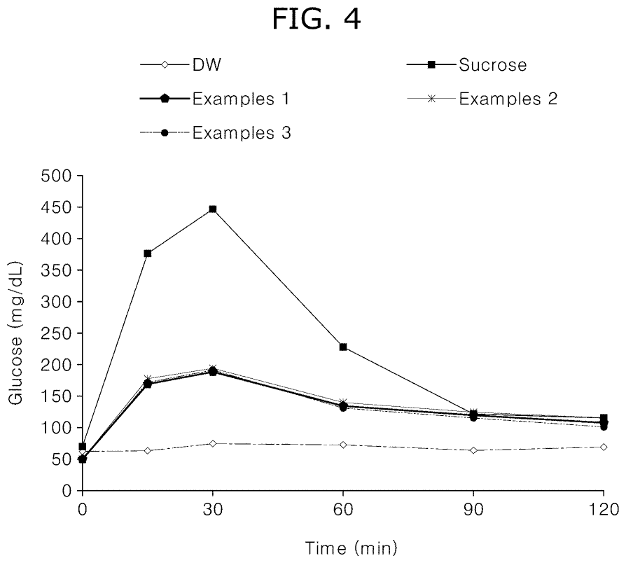

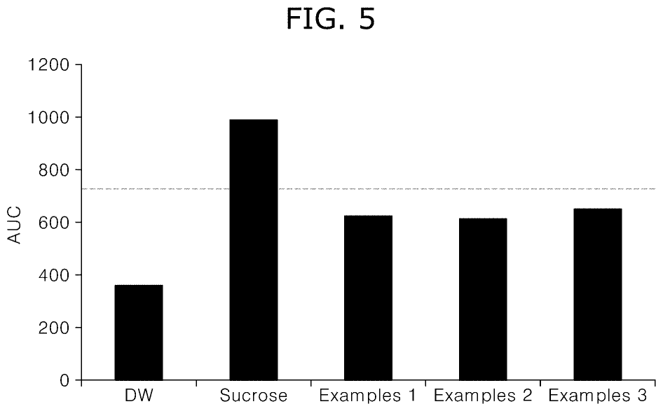

As a result, the palatinose syrups of Examples 1 to 3 showed similar effects of inhibiting the elevation of blood sugar level. Therefore, it has been confirmed that the syrup containing 20 to 22 wt % of palatinose based on the total solid contents had an excellent effect of inhibiting the elevation of blood sugar level, even though the composition ratios of other sugars, except for palatinose, were different (see FIGS. 4 and 5).

### Experimental Example 4

The degree of crystal formation was evaluated with respect to the palatinose syrups of Example 2, Example 3 and Comparative Example 1. Each of palatinose syrups was allowed to stand at room temperature and at low temperature (4° C.) for 6 months to confirm the crystallization of the palatinose syrup composition.

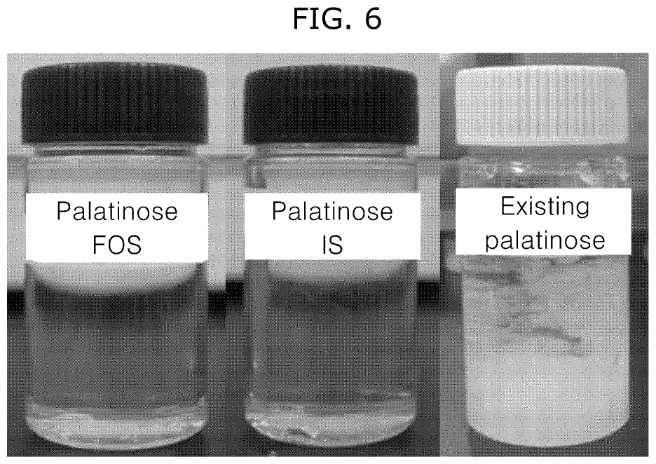

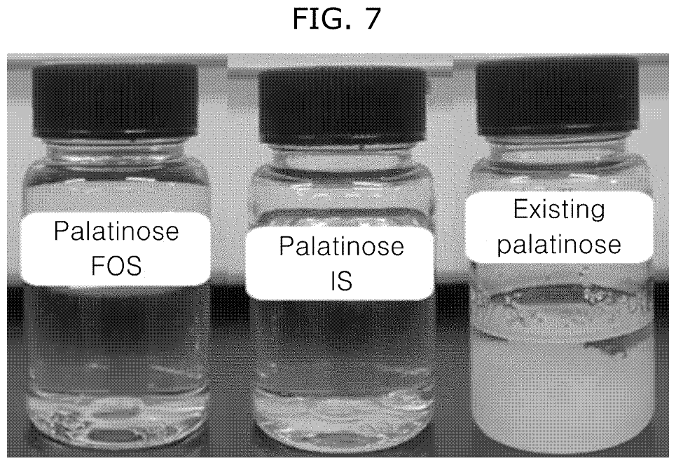

As a result, the crystallization of the palatinose syrups of Example 2 (Palatinose IS) and Example 3 (Palatinose FOS) was inhibited at both room temperature and low temperature, while it has been clearly observed with the naked eye that the palatinose syrup of Comparative Example 1 had crystals formed at room temperature and at a low temperature (see FIGS. 6 and 7, FIG. 6: the results of observing whether or not a crystal of the palatinose syrup is formed at room temperature, FIG. 7: the results of observing whether or not a crystal of the palatinose syrup is formed at low temperature).

1. Palatinose syrup comprising palatinose, glucose, fructose and trehalulose, wherein palatinose is comprised in an amount of 19 wt % to 22 wt % based on the total solid contents.

2. The palatinose syrup according to claim 1, characterized by comprising 80 to 250 parts by weight of glucose, 80 to 250 parts by weight of fructose, and 0.3 to 12 parts by weight of trehalulose, based on 100 parts by weight of palatinose.

3. The palatinose syrup according to claim 1, characterized by further comprising fructooligosaccharide.

4. The palatinose syrup according to claim 3, characterized by comprising 65 to 170 parts by weight of fructooligosaccharide, 65 to 170 parts by weight of sugar, 65 to 140 parts by weight of glucose, 20 to 70 parts by weight of fructose, and 0.3 to 12 parts by weight of trehalulose, based on 100 parts by weight of palatinose.

5. The palatinose syrup according to claim 1, characterized by further comprising sugar.

6. The palatinose syrup according to claim 5, characterized by comprising 300 to 420 parts by weight of sugar, 5 to 21 parts by weight of fructose, and 5 to 21 parts by weight of glucose, based on 100 parts by weight of palatinose.

7. The palatinose syrup according to a claim 1, characterized by having a sugar content of 69° to 78° BRIX.

8. The palatinose syrup according to claim 1, characterized in that the palatinose syrup does not contain crystals, precipitates and floaters at room temperature.

9. The palatinose syrup according to claim 1, characterized by having an inhibition activity of the elevation of blood sugar level.

10. A food composition for inhibiting the elevation of blood sugar level, comprising the palatinose syrup according to claim 1.

11. A method of suppressing the elevation of blood sugar level by administering to a subject the palatinose syrup according to claim 1.

12. A method of suppressing the elevation of blood sugar level by administering to a subject the palatinose syrup according to claim 2.

13. A method of suppressing the elevation of blood sugar level by administering to a subject the palatinose syrup according to claim 3.

14. A method of suppressing the elevation of blood sugar level by administering to a subject the palatinose syrup according to claim 4.

15. A method of suppressing the elevation of blood sugar level by administering to a subject the palatinose syrup according to claim 5.

16. A method of suppressing the elevation of blood sugar level by administering to a subject the palatinose syrup according to claim 6.
